(19) United States
(12) Patent Application Publication
(10) Pub. No.: US 2013/0130298 A1
(43) Pub. Date: May 23, 2013

(54) BLOOD PRODUCT MANAGEMENT METHOD USING RBC DEFORMABILITY-BASED METRICS

(71) Applicant: Blaze Medical Devices, LLC, Ann Arbor, MI (US)

(72) Inventors: Michael Tarasev, Pinckney, MI (US); Kenneth Alfano, Canton, MI (US)

(73) Assignee: BLAZE MEDICAL DEVICES, LLC, Ann Arbor, MI (US)

(21) Appl. No.: 13/726,272

(22) Filed: Dec. 24, 2012

Related U.S. Application Data
(63) Continuation-in-part of application No. 12/822,484, filed on Jun. 24, 2010.

(51) Int. Cl. C12Q 1/02

(52) U.S. Cl. CPC C12Q 1/02
USPC 435/29

(57) ABSTRACT

A method for using red blood cell deformability testing to improve management of blood product comprising RBC, the method comprising: generating deformability data for red blood cells corresponding to a respective unit of blood product; correlating the deformability data with red blood cell viability or efficacy based on any available direct or indirect in vivo performance data; obtaining a representation of quality for the respective unit of blood product; and based on the representation of quality assigning a relative rank to and/or timing a transfer of the respective unit in an inventory of stored blood product units.

### CROSS-REFERENCE TO RELATED APPLICATIONS

This is a US continuation-in-part application claiming the benefit of Ser. No. 12/822,484, filed Jun. 24, 2010, which is herein incorporated by reference in its entirety.

### FIELD OF THE INVENTION

This disclosure is in the field of medical methods. More particularly, it is in the field of methods for managing inventory of stored blood product, and specifically doing so by testing the RBC deformability of individual units of the blood product.

### BACKGROUND OF THE INVENTION

This section contains general background material, which is not necessarily prior art.

Red blood cell (RBC, erythrocyte) membrane properties are relevant to the cells' ability to perform their physiological function which is to travel the circulatory system and deliver oxygen to tissues. Deformability and fragility are two important and related membrane properties pertaining in some sense to cell “rigidity,” each of which can be measured by a number of different ways—and can have a range of possible applications spanning basic scientific research, blood product quality testing, and/or patient diagnostics.

In the United States, blood products containing RBC (e.g., RBC units, often known as stored “packed RBC” or “pRBC,” in addition to whole blood units or “WB”) are presently subject to a limited maximum shelf life, commonly of 42 days, and are typically managed by a “first-in-first-out” inventory approach (accounting otherwise for blood type, etc.). These facts, combined also with the recommendation that certain particularly vulnerable patient groups should receive fresher blood (e.g. for certain neonatal patients, blood of less than 7 days old is recommended—for “optimal” transfusion outcomes), constitute in effect a working presumption that storage time is the best indicator of blood quality—although notably, the usefulness of even this indicator remains the subject of much controversy. Other standards, like the 1% target maximum for in-bag auto-hemolysis during storage as well as the 75% target minimum for RBC to survive in vivo 24 hours after being transfused, get applied in statistically aggregated approaches and thus do not serve as quality indicators for particular product units in clinical practice.

### BRIEF SUMMARY OF THE INVENTION

This section briefly and non-exhaustively summarizes the subject matter of this disclosure.

The present disclosure describes a method for using red blood cell deformability testing to improve management of blood product comprising RBC, the method comprising: generating deformability data for red blood cells corresponding to a respective unit of blood product; correlating the deformability data with red blood cell viability or efficacy based on any available direct or indirect in vivo performance data; obtaining a representation of quality for the respective unit of blood product; and based on the representation of quality assigning a relative rank to and/or timing a transfer of the respective unit in an inventory of stored blood product units.

The scope of the invention is defined by the claims, which are incorporated into this section by reference. A more complete understanding of embodiments on the present disclosure will be afforded to those skilled in the art, as well as the realization of additional advantages thereof, by consideration of the following detailed description of one or more embodiments. Reference will be made to the appended sheets of drawings that will first be described briefly.

### BRIEF DESCRIPTION OF THE DRAWINGS

This section briefly describes the accompanying drawings for this disclosure. All drawings are for illustrative and explanatory purposes only and note intended to limit the present invention to any example embodiments depicted herein.

FIG. 1 is a flowchart depicting an embodiment of using deformability data to help improve management of blood product inventory over first-in-first-out (for a given type, etc.). Note that the alternative last-steps of either assigning or timing need not be mutually exclusive.

### DETAILED DESCRIPTION OF THE INVENTION

This section contains descriptive content for this disclosure. It is also to be understood that the terminology and phraseology used herein is for explanatory and exemplary purposes and not intended to be limiting, since the scope of the present invention is defined ultimately by the claims. This disclosure may comprise recapitulation and/or elaboration of select matter of earlier disclosure(s) from which it derives.

Cell deformability testing in general involves measuring how much or how easily cells can change shape or “deform” under stress—this broad concept is sometimes expressed with varying emphases, but all essentially maintain this core theme. (However, this concept is notably distinct from yet related to cell “fragility,” which instead looks essentially at how easily the cells lyse or rupture under stress—although such stress may happen to involve repeated cell deformations prior to such lysis occurring.) Deformability may also be called plasticity in some contexts, or referred to alternatively as a lack of rigidity or stiffness. Erythrocyte deformability can to some extent reflect red blood cells' ability to traverse/perfuse the human capillary network, so as to effectively transport oxygen without getting removed from circulation or obstructing microvascular flow. It can be measured in ways that reflect it as part of a collective blend of membrane-related and/or flow properties. This can be useful for various basic science and/or diagnostic applications.

Various approaches and techniques to assessing deformability of erythrocytes/RBC have been used over many decades for basic research studies and diagnostic inquiries, and commercial devices for measuring deformability have been developed and long pursued in such applications. It is often desirable to use techniques which are as physiologically-relevant as possible; that is, to simulate or otherwise well represent or correlate to kinds of stresses that RBC experience in vivo, so that its results can reflect how well the cells can perfuse bodily capillaries, for example. Some approaches can achieve this better than others, and sometimes the preferred test for deformability may be best ascertained empirically for each particular application; nevertheless, sometimes an approach having sub-optimal physiological relevance may simply be more practical because it is more readily available or clinically-adaptable, for example—or, perhaps because it is nevertheless sufficient for a given purpose.

Example approaches can include ektacytometry, optical tweezers, and pore filterability—among many other already-known or future-developed tests that involve an explicit or implicit measure of RBC deformability. Example values provided by such approaches may sometimes be designated as an elasticity index (EI) or a deformability index (DI), and results can reflect an aggregate of a sample being measured, or single cells individually (whether some or all of a given sample), or discernible sub-populations. It could also involve repeated testing, to ascertain any changes seen over multiple deformations.

This disclosure focuses on applying information pertaining to RBC deformability—however it gets measured—to inventory management of blood. The original Ser. No. 12/822,484 parent application described and enabled select approaches for using of RBC deformability information for evaluating quality or degradation thereof for specific units of blood product, and in particular addressed using such information to quantitatively reflect age-independent quality extents either at or since donation—and doing so for each respective unit tested (e.g., relative or absolute degrees of quality even among usable units, indicative of closeness to becoming unacceptable and/or potential degrees of acceptability, etc.), and not simply screening for negative outliers (e.g. classifying as “abnormal,” etc.). Moreover, such extents of quality could be used to reflect degrees of product acceptability, such as by selectively triaging those units deemed to be the “best” or least-degraded toward the most vulnerable patients. Note that the possible variations existing right at donation/collection are largely attributable to “donor-to-donor” differences (whether genetic or otherwise), which can also additionally be reflected (along with other factors) in subsequent/post-storage quality degradation. Moreover, even “same-donor” differences can occur from one donation to the next. That Ser. No. 12/822,484 parent application also contained disclosure to support certain other uses of RBC deformability information in the context of blood product quality assessments, some of which involve correlating such in vitro information to clinical outcomes (e.g., primary or secondary) or in vivo red cell performance. Correlative use of post-transfusion clinical data could optionally be combined with the aforementioned use of quantitative degrees of quality.

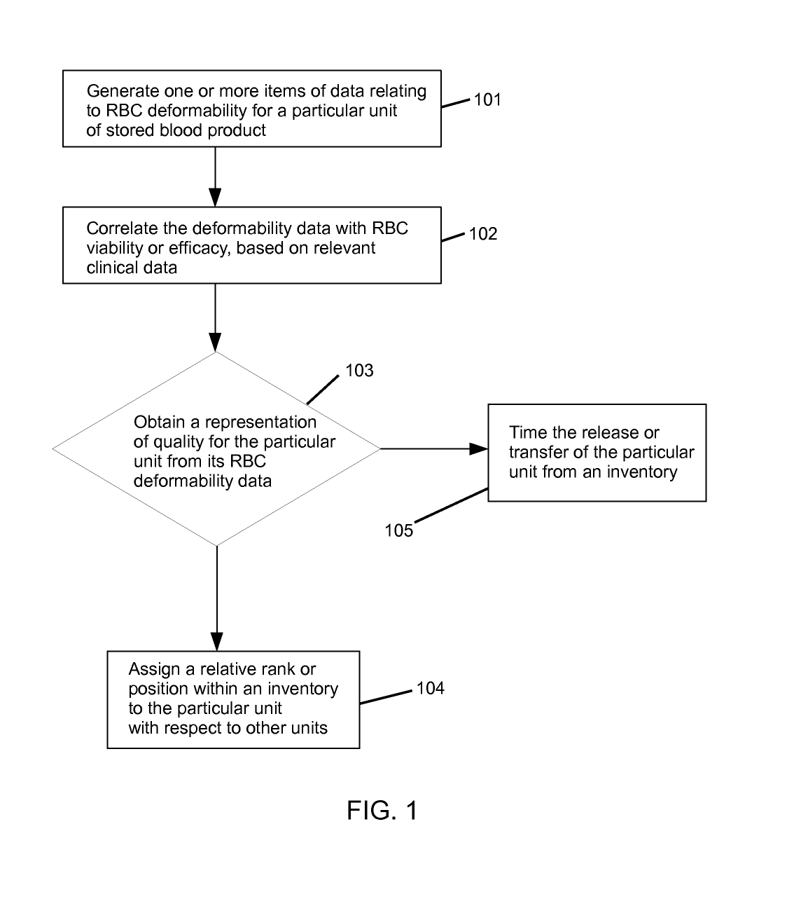

FIG. 1 depicts an embodiment of the present method, wherein in step 101 deformability data is generated for a particular unit of blood product containing RBC, which data is then correlated to clinical data in step 102, before being a basis for a representation of quality obtained in step 103. Steps 104 and 105 respectively involve either assigning a rank to or timing a release of the particular unit of blood product based on the preceding steps (note that those two concepts are not mutually exclusive).

Causes of quality differences that cannot be accounted for solely by storage time, nor fully controlled by standardizing storage conditions, can include for example: donor-to-donor differences (which can affect differences at the time of donation as well as differing propensities to degrade thereafter), including the fact that the range of “ages” of red cells within donors' bodies at the time of donation can vary from person to person (metabolic/physiological age); relatedly, same-donor differences from one donation to the next; also, conditions of production or storage that are within the tolerances and thus cannot be controlled away but nevertheless introduce variability; and of course, inherent variabilities in transportation conditions can involve more sources of “noise.” Notably, no such differences appear to have yet led to development of inventory management or utilization methods to account for “storage-time-independence” of RBC quality, and the focus (including associated controversies) remain centered predominantly upon the role of product “age.” Some possible sources of variability like bag material or storage solution could potentially be standardized. The distribution of cell ages (distinct from product/unit ages) within a given donor may be a factor in sub-populations within a sample exhibiting distinct characteristics; nevertheless, often it is desirable for a single value (e.g., an average via some deformability-based metric) to represent an overall sample, for the sake of simplicity. RBC membrane properties can also be used to ascertain which units are most amenable to certain manufacturing processes or storage conditions, by establishing a predictive correlation directly or indirectly between the in vitro property before subjection to said process or condition and relative in vivo performance.

Samples of RBC for testing can be taken directly from the main or “mother” bag of a fully processed and manufactured unit of blood product; alternatively, it could be taken from a pre-separated test segment typically attached thereto. Depending upon the manufacturing method for a given RBC or whole blood unit, as well as the type of bag structure, the test segment may have higher or lower ability to represent the true status of the main bag's contents. Another possibility is to draw a sample from a donor who donated or a prospective donor before he/she donates. (This latter possibility could be employed to “screen” potential donors according to their current and/or sustained erythrocyte properties even before deciding whether to collect at that time.)

The representation of quality obtained for a given RBC sample can simply be a selection of all or part of the deformability data itself—if such proves adequate—and the deformability data itself can be a single value measured or any other kind of relevant information (including indirect reflections of deformability, via observable effects thereof). It can also be performed concurrently with the correlating step, for example when the correlating step is a simple and implicit utilization of any then-accrued knowledge from clinical data regarding the dependency of RBC viability or efficacy upon one or more given measures of deformability.

While some device(s) other than a standard computer will be needed to generate one or more pieces of deformability data, the rest of the steps can potentially be performed either mentally and/or with a computer. For example, a physician could simply look at the deformability data for a given sample and use his/her knowledge of any then-published peer-reviewed studies linking similar deformability-related metrics to relevant post-transfusion clinical metrics—to in effect employ the data itself as a proxy for quality, upon which an inventory-related decision thus gets based; alternatively, the deformability data could be fed directly to a computer programmed to compare one or more aspects of the deformability data against any then-established correlations based on then-available historical clinical data (whether the clinical studies were done at the same facility or elsewhere is largely irrelevant, unless the user of the test wishes to continuously or immediately be updating the database of correlative data—which may happen to be the case with early adopters). Naturally, the assignment (or updates thereof) of an order/rank to a given unit or timing of its release/transfer may occur via incorporation in a hospital blood bank's computerized network—particularly if that's how it manages its inventory otherwise.

Clinical data upon which either explicit or implicit correlations can be made can include studies to substantiate associations between in vitro RBC membrane properties and in vivo RBC performance (e.g., RBC survival, or other relevant behavior such as tissue oxygenation) post-transfusion. It can also involve actual clinical outcomes like mortality or morbidity, although this requires enough statistical power to account for confounding variables. The FDA has a guideline that at least 75% of transfused RBC should survive in the patient for at least 24-hours post-transfusion (“recovery”), which can be tested in clinical studies with ⁵¹Cr labeling, but presently there is no test available to assess compliance of particular individual units in routine practice. Correlating deformability data/measurements with post-transfusion cell survival levels could provide some predictability as to whether this standard is likely to be met for a given unit—and even quantify how well. Another clinical metric could be tissue oxygenation (although this would require designing studies to ensure that the cells are being evaluated rather than patients' physiological compensation mechanisms, etc.). Clinical biomarkers known to be associated with certain clinical outcomes are another possible measure, as is the receiving patient's post-transfusion rise in hematocrit or hemoglobin.

Of course, as with most medical methods, optimal implementation involves the accumulation of copious output and associated correlations, so that with time the relevant characterizations become progressively more meaningful and accurate. Likewise, hurdles to broad general acceptance of any new medical/clinical method tend to require more conclusive evidence than is needed to begin piloting useful applications or even begin commercialization among early-adopters or opinion-leaders (provided of course that any requisite regulatory hurdles can be met, if applicable). Hence every new study will progressively tend to increase the reliability and value of the present invention. Furthermore, clinical validation may prove to involve correlations being made specifically for particular patient groups or conditions.

For applying the quality measurements toward improving blood product inventory management, any existing and known principles of operations research (OR), including supply chain or inventory management tools from other contexts can be employed here (one currently preferred is shortest remaining shelf life (SRSL)—already used in the field of perishable foods, as discussed below). Some in particular may prove more useful the further “up” the chain it is employed; for example, a blood collection center or a large centralized hospital system that supplies a network of smaller hospitals may have more opportunity to exploit better information. RBC units having “high” quality may be deemed better able to withstand certain product-modifying processes such as irradiation (used for immuno-compromised patients). Military applications could include selecting units appearing best-suited to withstand lengthy and/or harsh travel abroad. In many cases it is expectation that computational tools will aid in optimizing such applications.

As a simple example, moving units that appear to be degrading more rapidly to the “front of the line” would allow them to get used before becoming unacceptable (or avoidably less-acceptable), while holding back longer those units that are either degrading less rapidly and/or have a surplus of quality relative to other available units. One goal could be to optimize the net overall quality level dispensed across a given inventory (which could mean fewer RBC units being needed for some patients due to such patients receiving higher efficacy per unit, thus saving both blood product and procedural time and expense, in addition to avoiding some unnecessary complications); or in some cases, to simply target the “best” units for relatively earlier-timed release to especially vulnerable patients (e.g. neonates, critical-care, etc.). Appropriate modeling tools for decision analysis or management could include linear or dynamic programming, including single or multi objective functions, decision variables, constraints, etc. Initially any such optimization would simply take place within the existing 42-day maximum shelf life; however, if some measure for deformability becomes well established as indicative of RBC quality or suitability, the uniform shelf life rules could conceivably be modified to allow for some case-by-case consideration of more direct unit-specific testing.

In the case of blood management, the FIFO-based system of today results in a tension between some advocating for a shorter shelf-life (e.g., 28 days, or 14 days for certain patient groups) in order to reduce the number of units degrading unacceptably before use—versus maintaining the status quo in which a substantial percentage of units have been estimated to fail the aforementioned 24-hour/75% post-transfusion RBC survival standard. Recently published work by Atkinson, et al. discuss a combined approach blending FIFO and LIFO (last-in-first-out) as a possible way to better employ storage time of RBC product as a quality metric (versus FIFO alone), but this of course still uses storage time as the key metric—albeit in a more complex manner.

Shortest-Remaining-Shelf-Life (SRSL), also called Least-Shelf-Life-First-Out (LSFO), can complement/supplement or perhaps eventually replace First-In-First-Out (FIFO) in a similar fashion as it has is some cases for perishable foods. (Note that in the case of blood, “first in” refers to being first into post-collection storage, rather than when it first gets received by a particular hospital; hence, it could also be termed “oldest out first.”) In the case of food the focus of SRSL was on a given product's temperature history, but nevertheless the principle can carry over to any measurable “non-time” variable. For example, two food items of the same age but where one has been subjected to substantially more damaging heat will not be regarded with equal position in a queue; rather, the one with greater heat exposure, and thus likely faster “degrading,” will be prioritized for release while it is still of acceptable quality. In the absence of such a practice, expiration dates must be conservatively set so as to presume almost a “worst-case,” which causes inefficiencies when food that did not experience this worst-case must nevertheless be discarded prematurely. Thus, the cost and effort of tracking temperature history can be justified. The research and work of Wells and Singh are well regarded on this topic in the food distribution field—which can now be adapted to blood management opportunities, based on RBC deformability testing as a basis for deciding the relative or absolute “remaining shelf lives” of RBC units.

Appropriate algorithms can input stipulated parameters for minimum acceptable quality, projected demand levels or cycles, clinical outcome data, transit times (and perhaps conditions), possibly certain relevant costs, etc.—and then output guidance for ordering a release sequence or relative or absolute timing, or optimal points or intervals for conducting the tests, based on linear or more sophisticated quality-loss projections for given units (updatable upon each test, or other desirable intervals), as well as consideration of different initial quality levels. With sensitivity analyses, models can be used to assess various trade-off decisions—including the determination of when, on average, is the best time to initially test blood product units, and also the optimal testing frequency. Beyond appropriately accounting for ABO/Rh type distinctions (including measures to preserve respective minimum levels, and separate rare groups inventories as may be necessary), sub-inventories differentiated by leukoreduction-status or irradiation-status may be separately tracked, especially if they exhibit different patterns. Other more sophisticated model features could include considering only the “last” six (for example) units given to a high-volume transplant (for example) transfusion patient, in order to prioritize those units which will actually remain in such a patient post-surgery. Depending upon the sophistication of any modeling informing the assigning or timing decisions, appropriate hardware and software may be employed for constructing and/or implementing such models. Algorithmic and/or simulation based approaches preferably employ a computer, which can be any processing unit capable of running standard or customized programs to aid in establishing and/or implementing methods of product organization, utilization, scheduling, planning, tracking, logistics, forecasting, quality management or other operational matters.

Currently, “fresh” blood is first distributed to smaller/rural hospitals (in case it has to sit for a while due to small hospitals' lower blood use), but when it gets close to expiration it is taken to the large hospitals which have much higher throughput and thus are still able to utilize this blood prior to its expiration (thus reducing losses due to blood outdating). Hence the average age of blood transfused at larger hospitals tends to be older. With direct knowledge of unit-specific quality levels and/or rates (and/or accelerations), existing networks of inter-institutional blood product transit can be optimized as well as within a given facility itself—to improve the overall prospective quality (or efficacy) of blood units.

As various data is accumulated, the testing uses and implementation methods can become progressively more sophisticated. Answers to questions will emerge regarding how frequently testing should occur in a given inventory (or what factors influence this), how early should it start, the optimal age to measure at for projecting future degradation, and the marginal benefit of adding additional testing points during storage. The unit-to-unit differences in quality or degradation tendencies will likely affect the value of individually tracking quality of particular units to better manage decisions regarding routing/distribution—from collection centers and/or within or among hospital blood banks—or even inform other practices such as handling and transportation. With proper modeling, the net projected reduction in overall product degradation achievable by exploiting this potential can be estimated.

Other RBC membrane-related propert(ies) may closely correlate with some metric(s) for deformability, at least in some respect, in which case such could be employed as a proxy for deformability and thus still be deemed deformability-based (albeit perhaps indirectly so) insofar as a sufficiently close and clinically validated correlation exists to warrant it. For example, RBC morphological changes (e.g., sphericization) occurring during storage may prove associated with concurrent and/or impending deformability changes and/or progression theretoward. Furthermore, sometimes multiple relevant flow-related properties (e.g. agglomerability/aggregability, adhesiveness) along with deformability may be collectively reflected by certain in vitro tests; such tests would likewise be deemed deformability-based (albeit perhaps partially so). Other related membrane-based characteristics could pertain to vesiculation or microparticles.

The human spleen removes from circulation those RBC which have decreased deformability, and Deplaine et al. have shown that such sensing can be mimicked in vitro, via a micro-bead-based sorting device whereby the interbead spaces simulate the geometry of interendothelial slits in a spleen, thereby retaining poorly-deformable RBCs in accordance with the pressure being applied across the device. Another perfusion-based (microfluidic) mechanism for sorting out low-deformability RBCs was devised by Bitensky, et al., which was directed at stored blood for transfusion. They have also used in vitro microvascular networks as a way to test RBC deformability. As part of calibrating or validating or complementing these kinds of mechanisms, it may be important to also ascertain how much hemolysis is or is not occurring under whatever pressure is being applied—thereby in effect including a fragility test—even if the goal in such processes is to keep any induced hemolysis to a minimum. Fragility testing in the context of transfusion applications is a subject of the Ser. No. 12/690,916 “patent family.”

As indicated by the Ser. No. 12/822,484 parent of this application, tests of RBC “fragility,” especially direct ones, are expected to be superior to tests of “deformability” for determining certain kinds of cell attributes—for example, those dealing with cells' need to be able to be stressed on a sustained or repeated basis. Hence as a practical matter, fragility testing tends to utilize higher stress levels (i.e., so called “cell-lethal” levels) simply because it is generally ascertaining their limits of stressability—though this is not to say that fragility can't itself be a measure of pre-existing cell damage caused by a previous sub-lethal stress. Moreover, fragility (mechanical fragility or “MF” in particular) is best suited for multi-parameter (>1) stressing to give multi-dimensional (>2) profiles showing how hemolysis depends upon two or more stress variables such as extent/degree of intensity and extent/degree of duration (for a given type/kind) of stress. (Deformability testing could in a sense also be performed with multiple variable stress parameters; however, multi-dimensionality has unique value when profiling induced hemolysis.) The two broad properties of deformability and fragility may indeed prove useful in conjunction, as they both measure membrane properties albeit in different respects—or in some cases deformability may even serve as a proxy for fragility, or vice versa—although these notions would likely require highly application-specific empirical validation.

This disclosure is enabling to those of ordinary skill in the art, while maintaining adequate flexibility for reasonable adaptation. Moreover, those skilled in the art will appreciate variations of the examples and principles described herein, which are also intended to be within the scope of the present invention. Any references herein to “the invention” or the like are thus intended in this spirit.

We claim:

1. A method for using red blood cell deformability testing to improve management of blood product comprising RBC, the method comprising:
generating deformability-based measurement(s) of red blood cells corresponding to a respective unit of blood product;
correlating at least one of said measurement(s) with red blood cell viability or efficacy, based at least partially upon in vivo performance data from one or more units transfused previously;
obtaining from said at least one of said measurement(s) a representation of quality for said respective unit of blood product; and
assigning to said respective unit a rank or order relative to other units of blood product in an inventory thereof, with said rank or order being based at least partially upon said representation of quality of said respective unit relative to similar representations of quality obtained for said other units.

2. The method of claim 1, wherein said measurement(s) comprises a quantitative measurement characterizing said cells at least partially according to their deformability.

3. The method of claim 1, wherein said representation of quality consists essentially of a quantitative value consisting essentially of said measurement(s).

4. The method of claim 1, wherein said representation of quality reflects a rate of change of said viability or efficacy, said rate of change based upon two or more measurements of the same unit of blood product taken at different times of storage.

5. The method of claim 1, wherein the method is performed for multiple blood product units in an inventory, and wherein said representation of quality reflects quantitative intervals of quality beyond qualitative classification or ordinal position.

6. The method of claim 1, wherein said method is performed essentially when said respective unit is first collected from a donor, and said representation of quality reflects said respective unit's state prior to a significant storage.

7. The method of claim 1, wherein said red blood cells corresponding to a respective unit of blood product comprise a sample taken from said respective unit at some point in time after its post-collection processing and manufacturing.

8. The method of claim 7, wherein said red blood cells corresponding to a respective unit of blood product are taken from a peripheral test segment attached to said respective unit's main bag.

9. The method of claim 1, wherein said red blood cells corresponding to a respective unit of blood product comprise a sample taken directly from a donor or a prospective donor of said respective unit.

10. The method of claim 1, wherein the generating step is performed utilizing a commercially-available test whose results comprise a reflection of one or more aspects of cell deformability.

11. The method of claim 1, wherein the obtaining step or the assigning step is performed using a computer.

12. The method of claim 1, wherein the correlating step and the obtaining step are performed simultaneously.

13. The method of claim 1, wherein said in vivo performance data of said units transfused previously comprises post-transfusion cell survival data.

14. The method of claim 1, further comprising during or after the assigning step, designating said respective unit to be subjected to irradiation, based at least partially upon said rank or order, wherein said respective unit had not yet already been irradiated.

15. The method of claim 14, further comprising during or after the assigning step, allocating said respective unit to be transfused to a neonatal or immuno-compromised patient, based at least partially upon said rank or order.

16. A method for using red blood cell deformability testing to improve management of blood product comprising RBC, the method comprising:
generating deformability-based measurement(s) of red blood cells corresponding to a respective unit of blood product;
correlating at least one of said measurement(s) with red blood cell viability or efficacy, based at least partially upon clinical data linking post-transfusion RBC survival or behavior in patients to pre-transfusion RBC deformability;
obtaining from said at least one of said measurement(s) a representation of quality for said respective unit of blood product; and
timing a release or transfer of said respective unit from an inventory of stored blood product, based at least partially on said representation of quality, said release or transfer not being an immediate discard.

17. The method of claim 16, wherein said release or transfer is from a hospital blood bank inventory to a patient's physician or other healthcare practitioner for use in transfusion.

18. The method of claim 16, wherein said release or transfer is from a blood collection center's inventory to a hospital's inventory or from a hospital's main inventory to an inventory of an affiliated satellite facility.

19. The method of claim 16, wherein the timing of said release or transfer is conducted partly according to patient condition.

20. The method of claim 16, wherein a first unit which is older than a second unit of same ABO type and Rh factor is purposely held in inventory until after said second unit is released, and wherein said representation of quality for said first unit indicates a lower level or rate of quality degradation compared to said second unit, and wherein the timing of said release or transfer is not based wholly on an approach of FIFO or LIFO or a combination of FIFO and LIFO.
